(19) United States
(12) Patent Application Publication
(10) Pub. No.: US 2003/0208125 A1
(43) Pub. Date: Nov. 6, 2003

(54) Fundus Camera

(76) Inventor: Rodney Dennis Watkins, Thebarton (AU)

Correspondence Address: EDWARD W CALLAN, NO. 705 PMB 452, 3830 VALLEY CENTRE DRIVE, SAN DIEGO, CA, 92130, US

(21) Appl. No.: 10/408,421

(22) Filed: Apr. 7, 2003

(30) Foreign Application Priority Data
Apr. 9, 2002 (AU) PS1583

(51) Int. Cl. A61B 6/00

(52) U.S. Cl. 600/473; 600/476

(57) ABSTRACT

An eye fundus camera (1) having an imaging axis (68) between an objective lens assembly (25) and a sensor arrangement (66), an illumination injection arrangement (80) between the objective lens assembly and the sensor arrangement and an illumination apparatus (62) to provide illumination to the illumination injection arrangement. The illumination apparatus includes a single light source (80) adapted to provide both infra-red and visible light and a selector (82) to select either the infra-red or visible light as well as a xenon flash lamp (85). Viewing can be by eyepiece (27) or screen or by photography. The camera can be mounted onto an ophthalmic slit lamp (2) assembly.

### FIELD OF INVENTION

This invention relates to an optical instrument for the viewing and imaging of the fundus of the eye and an arrangement for using such a device.

### BACKGROUND OF THE INVENTION

Cameras are known which can be used to view and image the fundus of the eye.

In such cameras it is desirable to have one mode of viewing using visible light and another mode of viewing using long wavelength visible or infrared light. Visible light causes the pupil of the eye to constrict and so if only visible light mode is available then drugs must be used to cause the pupil to dilate for viewing and imaging. Long wavelength visible or infrared light does not cause the pupil to constrict and hence a method of viewing of the eye illuminated with long wavelength visible or infrared for the purposes of image selection is desirable. The problem exists, however, that an image sensor sensitive to infrared light must be used as the naked eye cannot view infrared. Visible light can be used for the actual stage of flash photography of the fundus of the eye because the flash duration is very short and the image is obtained before the pupil of the eye has constricted.

A fundus camera or imager such as disclosed in U.S. Pat. No. 5,668,621 has two different sources of illumination, one used for infra-red and one used for visible light, and two different image sensors, one for each type of illumination. This means that there are required to be more extensive optics and hence it is a larger device and a more expensive device.

Similarly U.S. Pat. No. 5,543,865 discloses a device which has two different sources of illumination, one for infra-red light and one for visible light, and two different image sensors, one for the infra-red light and one for the visible light.

U.S. Pat. No. 4,572,627 again uses separate infra-red and visible light sources and separate image sensors.

It is the object of this invention to provide a simpler type of fundus camera which uses a single light source for both infra-red and visible light for the purposes of image selection and a single image sensor.

For some diagnostic purposes it is desirable that illumination of the eye be achieved by light that does not include any red light and hence it is a further object to provide a camera which has a red light removal arrangement.

To make a fundus camera which is useful for a practitioner to use in a consulting room it is desirable that it does not take up more space than necessary and hence it is a further object that the fundus camera be integrated with existing consulting room equipment.

### BRIEF DESCRIPTION OF THE INVENTION

In one form therefore, although this may not necessarily be the only or broadest form, the invention is said to reside in an eye fundus imaging apparatus having a digital camera sensor adapted to receive an image of the fundus of the eye wherein the digital camera sensor is adapted to be responsive to both infra-red and visible spectral bands utilised in the apparatus.

In a further form the invention may be said to reside in an eye fundus imaging apparatus having a sensor arrangement, an illumination arrangement including a single light source adapted to provide both infra-red and visible light and a selector to select either the infra-red or visible light and wherein the sensor arrangement is a single digital camera sensor adapted to be sensitive to both visible and infra-red light. A suitable electric motor may be provided to actuate the selector to select either the infra-red or visible light.

The single light source may be a halogen light source which provides both the visible and infra-red light. A fixed filter may be used to remove ultraviolet emitted from the light to protect a patient's eye. For instance, incident wavelengths below 400 nm may be removed.

In an alternative form the invention may be said to reside in an eye fundus imaging apparatus having an imaging axis between an objective lens assembly and a sensor arrangement, an illumination injection device between the objective lens assembly and the sensor arrangement and an illumination apparatus to provide illumination to the illumination injection device wherein the illumination apparatus includes a single light source adapted to provide both infra-red and visible light and a selector to select either the infra-red or visible light.

Preferably the single light source is a halogen light source which provides both the visible and infra-red light and the selector includes selectable filters to transmit either visible or infra-red light. The halogen light may be a 12 volt 25 watt lamp.

There may be further included a flash lamp close to or at an optical conjugate of the halogen light source. The flash lamp may be a xenon lamp. The flash lamp is used to actually take photographs of the fundus of the eye but, as discussed above, the flash duration is very short and the image is obtained before the pupil of the eye has constricted. As the flash lamp is at the optical conjugate to the halogen light source the same optical system can be used for the flash photography.

The illumination injection device may be a prism or a mirror.

The illumination injection device is preferably offset from the imaging axis and set to provide an illumination axis which is at a slight angle to the imaging axis and to focus the illumination approximately at the pupil of a patients eye but not at the imaging axis for efficient illumination of the fundus of the patients eye. Preferably the illumination injection device is positioned outside the image area between the objective lens assembly and the sensor arrangement.

Optionally there may be further included a relay lens assembly and an imaging lens assembly between the objective lens assembly and the sensor arrangement and the illumination injection device may then be positioned between the relay lens assembly and the imaging lens assembly and offset at a slight angle from the imaging axis. Preferably the illumination injection device is positioned outside the image area between the relay lens assembly and the imaging lens assembly.

There may be further included a movable mirror between the imaging lens assembly and the sensor arrangement, the movable mirror when extending into the imaging axis being arranged to direct an image from the patient's eye to an eyepiece on an eyepiece axis. The eyepiece can be used by a practitioner for image selection when the illumination apparatus is in visible light mode. A suitable electric motor may be provided to move the movable mirror into the imaging axis as required.

The eyepiece axis may include inverting prisms so that an upright image of the eye can be viewed in the eyepiece.

There may be further included a fixed filter on the illumination axis to remove ultraviolet radiation emitted from the light source to protect a patient's eye from incident wavelengths below 400 nm. The ultraviolet radiation can be emitted from both the halogen lamp and the xenon lamp.

There may be further included when required a red free filter or cyan subtractive filter in the illumination axis.

Preferably the sensor arrangement is a single digital camera sensor adapted to be sensitive to both visible and infra-red light.

There may be further included means to electronically adjust the position of at least one lens of the objective lens assembly, the relay lens assembly or the imaging lens assembly whereby to adjust the imaging apparatus for variations in the refractive error of the subject eye under investigation.

In a preferred embodiment the imaging lens assembly is adjustable to adjust the imaging apparatus for variations in the refractive error of the subject eye under investigation. Adjustment may be by moving the imaging lens assembly along the imaging axis. The eye fundus imaging apparatus may include electronic controls which include selectable positions of the imaging lens to adjust for variations in the refractive error of the subject eye under investigation. This automatic focussing may be set to adapt for a patient's refractive error range of from for instance −20D to +15D. A suitable electric motor may be provided to adjust the position of the imaging lens assembly for this purpose.

In an alternative arrangement the objective lens assembly is adjustable to adjust the imaging apparatus for variations in the refractive error of the subject eye under investigation in a similar manner to that discussed above.

In a still further form the invention may be said to reside in an eye fundus imaging apparatus having an imaging axis between an objective lens assembly and a sensor arrangement, an imaging lens assembly between the objective lens assembly and the sensor arrangement and an illumination injection device positioned between the objective lens assembly and the imaging lens assembly and offset from the imaging axis and set to provide an illumination axis which is at a slight angle to the imaging axis and to focus approximately at the pupil of a patients eye, a lamp to provide illumination to the illumination injection device and a selector to select either the infra-red or visible light, wherein the lamp includes a single light source adapted to provide both infra-red and visible light, wherein the selector includes selectable filters to transmit either visible or infra-red light and wherein the sensor arrangement is a single digital camera sensor adapted to be sensitive to both visible and infra-red light.

Preferably the single light source is an halogen light source which provides both the visible and infra-red light.

There may be further included a flash lamp at a focus point of the halogen light source. The flash lamp may be a xenon flash lamp.

In some circumstances it is desirable to change the camera from looking at the retina of the eye to look at the anterior of the eye so that the camera can be accurately aligned to the anterior using long wavelength visible or infrared radiation. For this purpose there may be further provided an adaptor lens assembly on the imaging axis between the objective lens and the imaging lens and this adaptor lens may be moveable between a position on the imaging axis when it is required to a position outside the imaging axis when it is not required. Hence when infrared viewing is selected the adaptor lens assembly may be adapted to automatically move into the imaging axis. A suitable electric motor may be provided to move the adaptor lens assembly. The infrared illumination may be provided by suitable LEDs at the front of the camera.

In a still further form the invention may be said to reside in an eye fundus imaging apparatus having an imaging axis between an objective lens assembly and a sensor arrangement, an imaging lens assembly between the objective lens assembly and the sensor arrangement and an illumination injection device positioned between the objective lens assembly and the imaging lens assembly and offset from the imaging axis and set to provide an illumination axis which is at a slight angle to the imaging axis and to focus approximately at the pupil of a patients eye, a movable mirror between the imaging lens assembly and the sensor arrangement, the movable mirror when extending into the imaging axis being arranged to direct an image from the patient's eye to an eyepiece on an eyepiece axis, a lamp to provide illumination to the illumination injection device and a selector to select either infra-red or visible light from the lamp, a flash lamp close to or at an optical conjugate to the lamp to provide illumination for photography, wherein the lamp includes a single light source adapted to provide both infra-red and visible light, the selector includes selectable filters to transmit either visible or infra-red light and the sensor arrangement is a single digital camera sensor adapted to be sensitive to both visible and infra-red light.

There may be further included a fixed filter on the illumination axis to remove ultraviolet emitted from the light source to protect a patient's eye from incident wavelengths below 400 nm. The ultraviolet can be emitted from both the halogen lamp and the xenon lamp.

There may be further included when required a red free filter or cyan subtractive filter in the illumination axis. A suitable electric motor may be provided to move the red free filter or cyan subtractive filter as required.

The eye fundus imaging apparatus of the invention may be adapted to be mounted to an ophthalmic slit lamp assembly thereby making it usable with existing equipment in a practitioners consulting rooms. The fundus camera can be mounted onto the tonometer adaptor on the ophthalmic slit lamp assembly and be to aligned with the patient's pupil using the joystick and height adjustment of the ophthalmic slit lamp assembly in an alignment mode and arranged so that at that stage the camera is is centred on the patient's pupil and the correct distance for fundus photography. Focus for the fundus of the eye requires for adjustment to compensate for the refractive error or dioptre of the patients eye. In one embodiment of the invention the compensation for the refractive error or dioptre of the patients eye may be done automatically by settings on the control panel of the camera.

There may be further provided a remote manual or foot operated switch arrangement for the fundus camera of the present invention thereby providing a practitioner with ease of operation of the camera.

The use of a single digital camera for both infra-red and visible light reduces the cost of the imaging apparatus according to this invention which will enable more practitioners to have such a device and hence more people can be examined.

By injecting the illumination at a slight angle to the imaging axis but using the same lens system to transmit the illumination to the patient's eye there is a simpler optical system provided and there is the advantage that reflections from the cornea and lens of the patients eye are not directed back along the imaging axis into the sensor. That is, the use of illumination at a slight angle to the image axis reduces ghosting or generation of images of the light source from the patients eye along the imaging axis.

### BRIEF DESCRIPTION OF THE DRAWINGS

This then generally describes the invention but to assist with understanding reference will now be made to the accompanying drawings which show a preferred embodiment of the invention.

In the Drawings:

FIG. 1 shows one view of an eye fundus camera according to one embodiment of the present invention from one side and mounted onto a slit lamp assembly;

FIG. 2 shows a view from the other side of the eye fundus camera of FIG. 1;

FIG. 3 shows the eye fundus camera of FIG. 1;

FIG. 4 shows a rear control panel of an eye fundus camera of FIG. 3;

FIG. 5 shows a schematic view of the optical path of one embodiment of an eye fundus camera according to the invention.

FIG. 6 shows a selectable filter arrangement suitable for the eye fundus camera of the present invention;

FIG. 7A shows a spectral response of the pixels of an image sensor suitable for use in the present invention.

FIG. 7B shows the sum of the spectral responses of the pixels making up the image sensor shown in FIG. 7A.

FIG. 8 shows a schematic view of an alternative embodiment of the optical path of an eye fundus camera according to the invention.

### DESCRIPTION OF PREFERRED EMBODIMENTS OF THE INVENTION

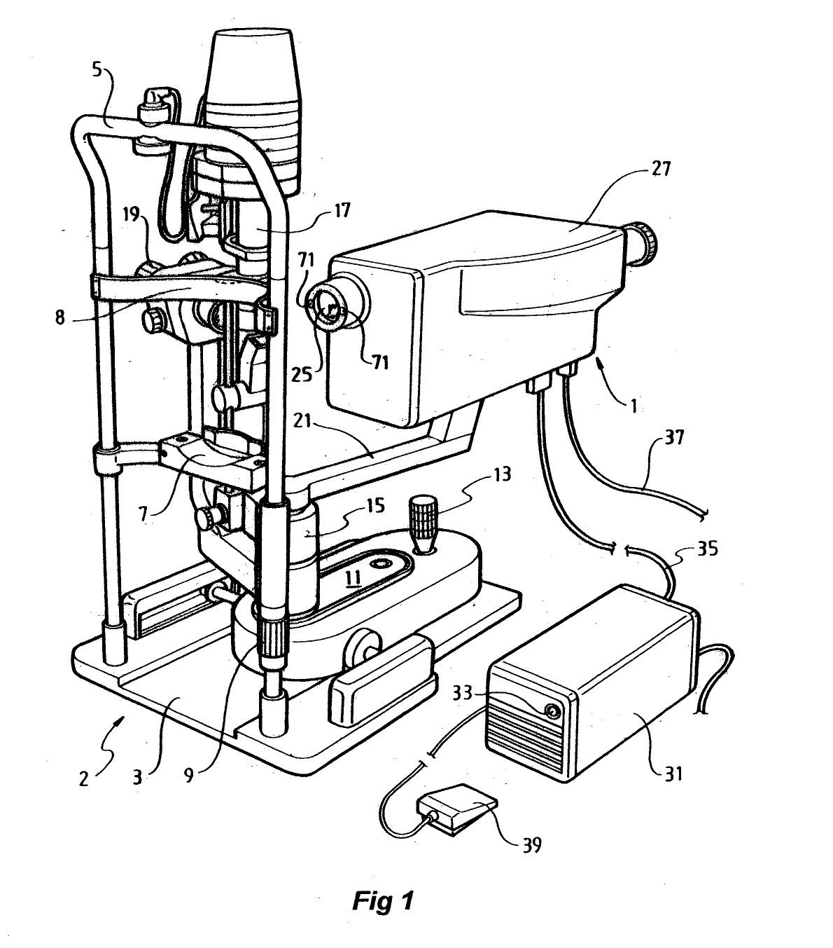

Now looking more closely at the drawings and in particular FIGS. 1 to 4 it will be seen that the eye fundus camera generally shown as 1 is mounted on to an ophthalmic slit lamp assembly generally shown as 2.

The ophthalmic slit lamp assembly 2 includes a base 3 with a head frame 5 at one end. The head frame includes a chin rest 7 and a forehead rest 8 for a patient. The height of the chin rest 7 on the head frame 5 can be adjusted by means of an adjustment screw 9 on the head frame 5.

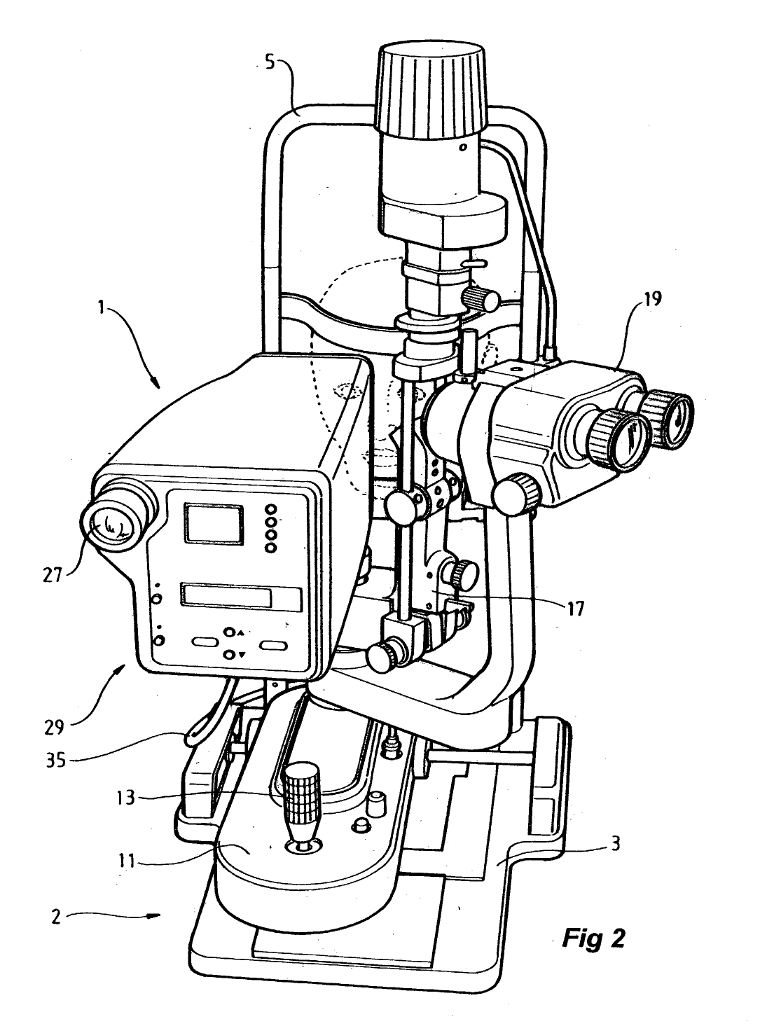

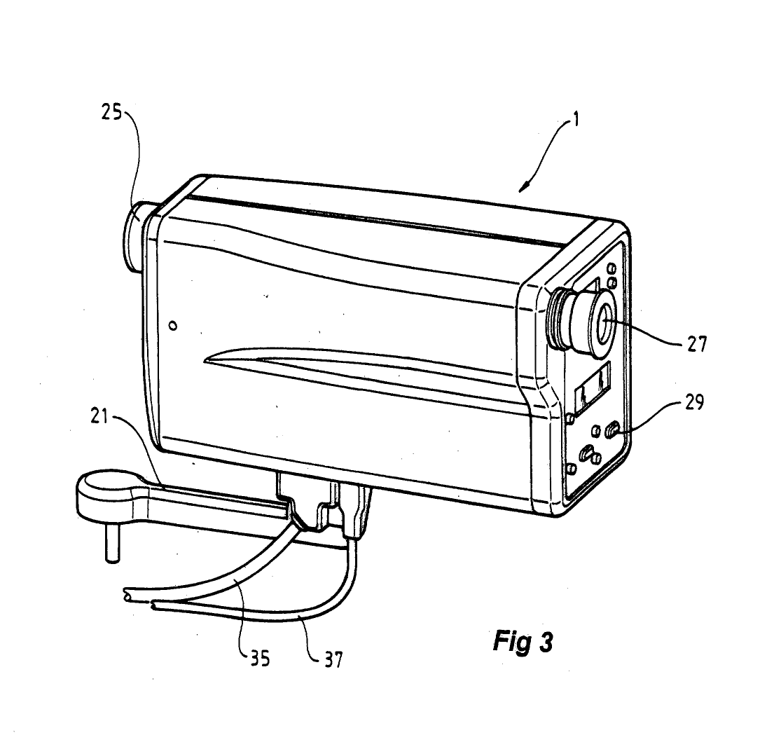

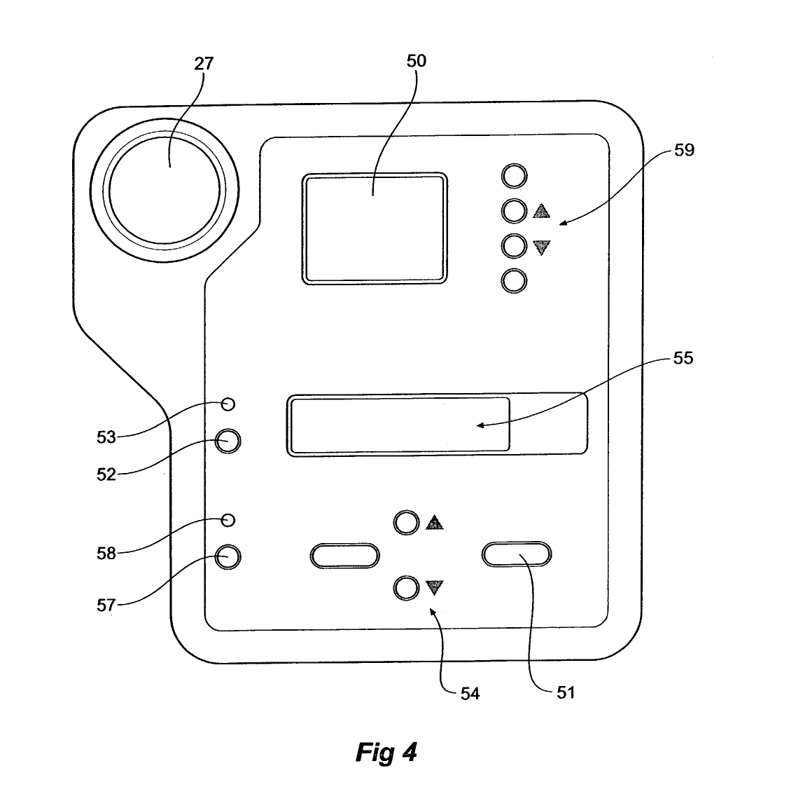

Also on the base 3 is an instrument mount 11. The position of the instrument mount 11 with respect to the base 3 may be varied by means of a joystick 13. Movement of the joystick 13 can adjust the position of the instrument mount 11 on the base both fore and aft and from side to side. On the instrument mount 11 is a post 15 upon which are independently rotationally mounted a lamp assembly 17 and a microscope assembly 19 as well as a support frame 21 for the fundus camera 1. The support frame 21 for the fundus camera 1 is mounted onto the tonometer adaptor on the ophthalmic slit lamp assembly. Each of the eye fundus camera 1, the microscope 19 and the slit lamp 17 can be independently swung about the mounting post 15 on a vertical axis to be positioned in front of a patient's eye as required by the optician. Generally the eye fundus camera 1 has an objective lens assembly 25 extending from one end and eye piece 27 extending from the other end. The control panel 29 on the eye fundus camera as can be seen in FIGS. 2 and 3 will be discussed in more detail with respect to FIG. 4.

On the objective lens assembly 25 there is positioned two red LEDs 71 which are used in alignment mode as will be discussed later to illuminate the anterior of the eye so that the camera can be correctly aligned with the eye before photography.

As can be seen particularly in FIG. 1 adjacent the base 3 is a power supply 31 for the eye fundus camera with a power switch 33 and a power cable 35 extending to the eye fundus camera 1. A remote switch 39 to actuate functions of the eye fundus camera can be operated by hand or by foot and can particularly can be used for hands-free operation of the camera where a practitioner has his hands full with a patient. A lead 37 extending from the camera can be connected to a computer for downloading, recording and printing of the digital photographs which have been taken using the eye fundus camera. The lead 37 preferably has a USB type connection.

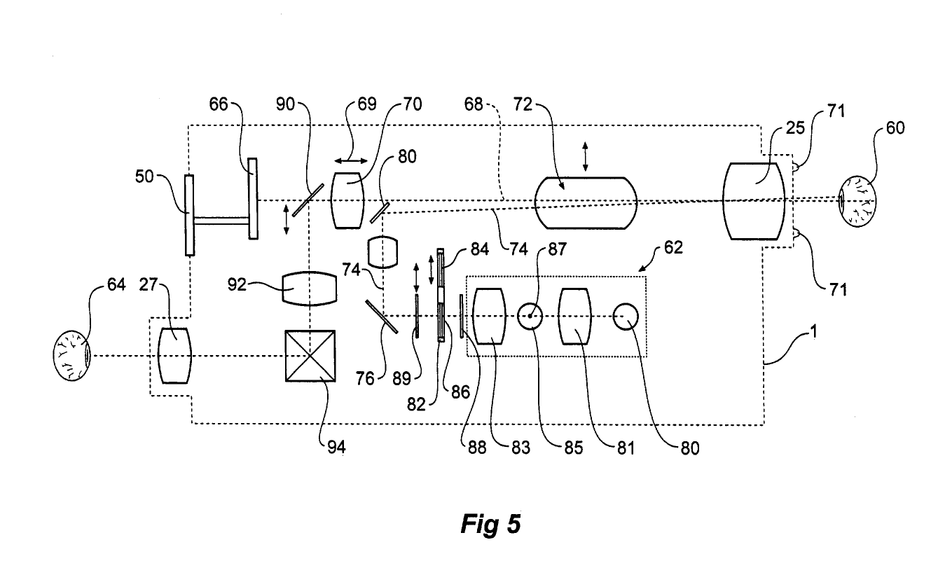

FIG. 4 shows a rear view incorporating the control panel 29 of the eye fundus camera according to this embodiment of the invention. On the control panel 29 there is the eye piece 27 and controls for the eye fundus camera. A preview screen 50 enables a view of the digital image being seen through the objective lens as will be discussed with respect to FIG. 5.

Also on the rear panel of the eye fundus camera 1 is a power button 52 and power “on” indicator light 53, menu controls 54 and a menu display screen 55. A red-free filter selector button 57 is also on the rear panel and an “on” indicator light 58 for when the red-free filter is being used. Advanced camera controls 59 are also on the back panel. The advanced camera controls are used for camera setting and image handling functions such downloading of images.

The menu display screen 55 and menu controls allow selection of the various functions of the camera as follows:

1. Patient's Refractive Error.

By entering the patients refractive error, the camera 1 is automatically focussed for the sharpest image. For astigmatic eyes, the “best sphere” power (the midpoint between the two astigmatic line powers) should be used. Cylinder powers of less than 2D will have little effect on image quality.

2. Viewing Mode

There are three ways to view the fundus:

i. through the eyepiece 27 in visible light (DV/VIS),

ii. via the preview screen 50 in visible light (CAM/VIS), or

iii. via the preview screen 50 in near-infrared radiation (CAM/RED).

Note: The visible light modes require pupil dilatation.

In addition, an alignment mode can be selected to view the anterior eye with infrared illumination, for easy orientation in the patient's pupil. Illumination of the anterior of the eye is by means of red LEDs 71 positioned either side of the objective lens assembly 25.

In the alignment mode the image of the eye is viewed on the preview screen 50. In the alignment mode the joystick 13 on the ophthalmic slit lamp assembly 2 is used to vary the position of the fundus camera 1 until the anterior of the eye is in focus at which stage the camera is at the correct distance from the eye for fundus photography. Adjustment for the refractive index of the eye may be by settings provided on the control panel as will be discussed below. The alignment mode can also be used to photograph the anterior of the eye using illumination of the eye from the slit lamp 17.

3. Lamp Brightness Indication:

The halogen lamp 80 generates both visible light and infrared energy. The level can be cycled through nine levels using the Up and Down buttons of the menu controls 54, or the lamp can be turned off (Level 0).

4. Flash level

The xenon flash level can be selected for the correct image exposure. Variations in pupil size and fundus pigmentation will affect the exposure.

The advanced camera controls 59 allow for image manipulation such allowing for the images captured by the camera to be previewed on the preview screen 50. They also allow many of the functions to be carried out at the camera, such as allowing the deletion of selected images. Other functions of the advanced camera controls are to change the image capturing performance of the digital camera. These are preset, and would normally not need adjustment by the user.

The image capture button 51 is also on the control panel 29. When the image capture button 51 is pressed, the image shown on the preview screen 50 is captured with the following steps—:

i. the halogen lamp 80 is switched off

ii. the infrared protection filter 84 is positioned in front of the xenon flash tube

iii. the xenon flash lamp 85 is activated.

The image captured is shown in the preview screen 50. It will take a few moments to process the image, depending upon the various image format selected. For example, a lossy JPEG image will take longer to process than a loss-less DPCM image (although the loss-less image will use considerably more storage space in the Internal Camera Memory) During this processing time, the text “IMAGE PROCESSING” appears on the menu display screen 55.

FIG. 5 shows a schematic optical arrangement for the eye fundus camera of the present invention.

The eye fundus camera 1, shown in FIG. 5 by the dotted line, is used to view a patient's eye 60 after it has been illuminated by an illumination arrangement 62. Viewing can either be visually through the eye piece lens 27 to an operator's eye 64 or on a preview screen 50 in which the optical signal is received on a digital sensor 66 and converted into a digital image which can be stored and/or displayed.

In the eye fundus camera 1 an imaging axis 68 includes an objective lens assembly 25 and an imaging lens assembly 70 which directs light onto the digital camera sensor 66. The position of the imaging lens assembly 70 may be adjusted, as shown by arrow 69, to allow for the existing refractive index of a patient's eye so that an accurate image of the retina of the eye can be obtained. The adjustment can be manual but is preferably automatic in response to settings made via the control panel 29. An adaptor lens 72 can be moved in or out of the objective axis 68 to adjust the point of focus from the retina of the patient's eye 60 to the pupil of the patient's eye so that the camera can be accurately aligned in the pupil using long wavelength red radiation.

Illumination from the illumination assembly 62 is provided on an illumination axis 74 which is reflected first on mirror 76, through illumination lens assembly 78 and then another mirror 80. The mirror 80 is off-set from the imaging axis 68 and directs light through the objective lens 25 to illuminate the patient's eye 60. The illumination axis 74 after the mirror 80 is at a slight angle to the imaging axis 68 so that less reflections of the light source are obtained from the pupil of the patient's eye which may cause a problem with unwanted light on the digital sensor 66.

The illumination arrangement 62 includes a halogen light source 80, a first condensing lens 81 and a second condensing lens 83. The halogen light source 80 is used for either visible or infrared illumination of the eye for alignment of the camera and visual observation using the digital camera as will be discussed later. For flash photography a xenon flash lamp 85 is used as will be discussed later. The xenon flash lamp 85 is placed between the first condensing lens 81 and the second condensing lens 83 at or near a conjugate point of the halogen light source 80. As both the halogen lamp 80 and the xenon lamp 85 can produce harmful ultra-violet radiation a permanent UV filter 88 is provided in the illumination axis. As the camera, according to this invention, is adapted for both infrared and visible light viewing a filter assembly 82 includes two filters on a movable base so that either infrared light using filter 84 can be passed or visible light using filter 86 can be passed but not both, as required. A cyan subtractive or red-free filter 89 may also be provided for particular photography when a user wishes to have no red in the light source.

Between the imaging lens assembly 70 and the digital camera sensor 66 is a movable mirror 90 which can be swung into the imaging axis to direct light, which would normally have passed to the digital camera sensor 66, through an eye piece lens assembly 92 and inverting prisms 94 to the eye piece lens 27. The inverting prisms put the image of the fundus of the eye into a correct orientation for viewing by a practitioner. This enables a practitioner to directly view the fundus of the eye when visible light is being used.

Further illumination using red or infrared illumination may be provided using one or more red or infrared LEDs 71 on the objective lens 25. These are used to illuminate the pupil of a patient's eye during alignment procedures as discussed earlier.

The camera according to this invention can have an alignment mode, three modes of viewing the fundus and two modes of photography.

It is necessary to position the fundus camera accurately in the patient's pupil plane and also at the correct distance from the patient's eye in order to produce an image of the fundus which fills the field of view and is free from reflections. The first mode of viewing allows the operator to correctly position the fundus camera by adjusting the slit lamp assembly height and joy stick controls. By viewing the patient's pupil directly the operator can centre the fundus camera in the patients pupil. When the image of the patient's pupil is in sharp focus the fundus camera is at the correct distance from the patients eye for fundus photography.

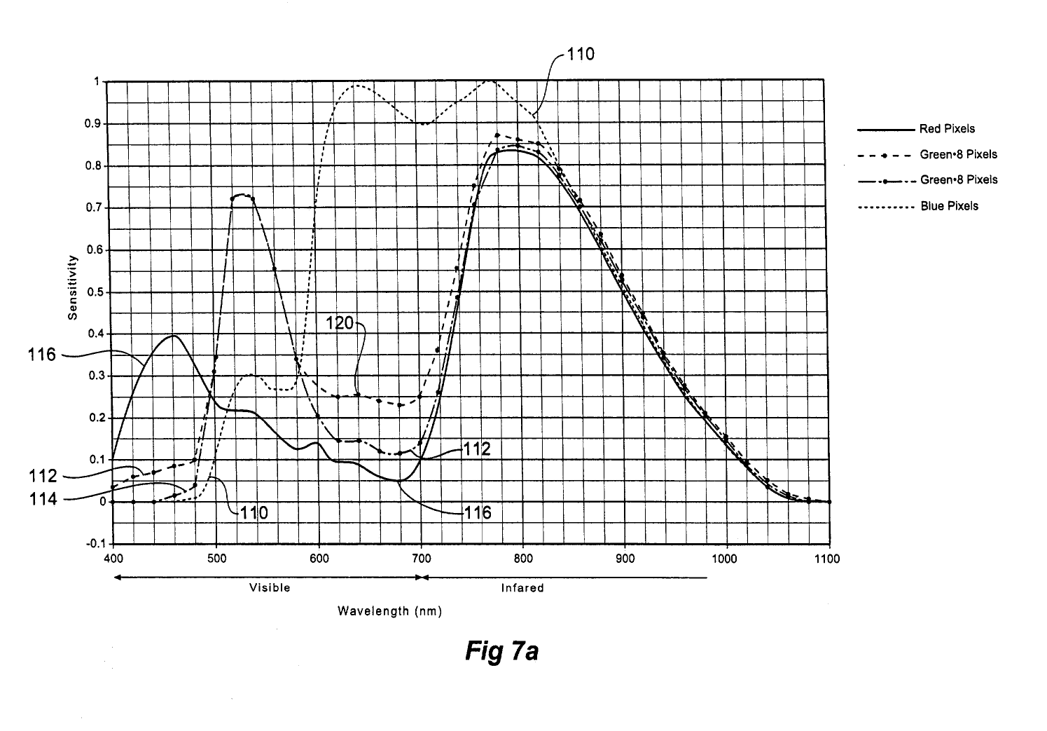

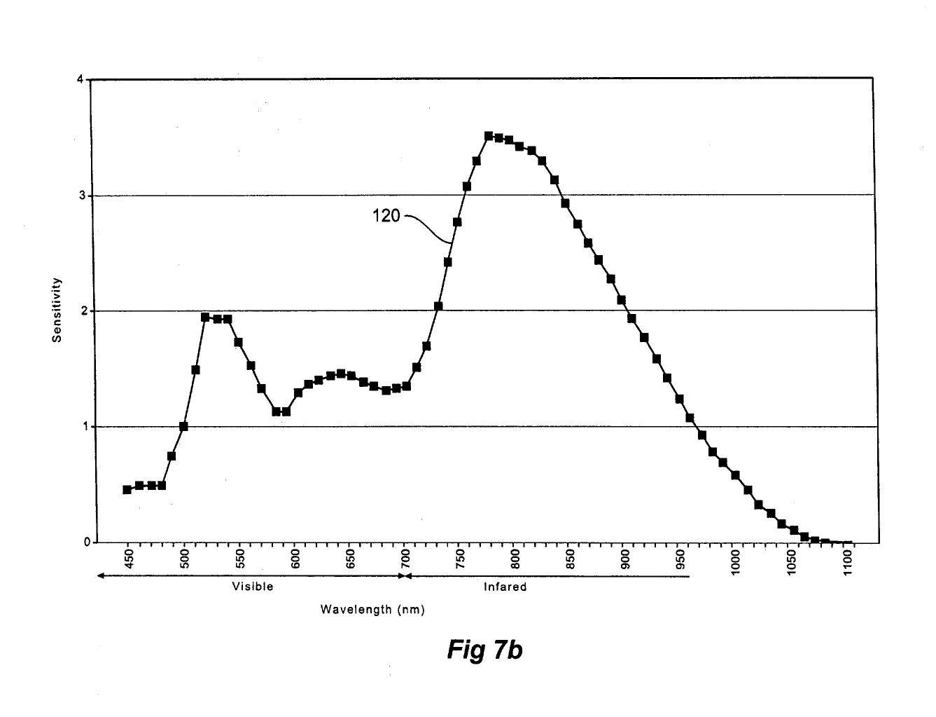

In the first mode of viewing red or infrared LEDs mounted on the objective lens are switched on to illuminate the eye and the digital camera sensor 66 which is sensitive to a wide range of light from visible to infra-red as will be discussed in relation to FIGS. 7A and 7B is used to collect the image and the resultant images shown on a display screen 50 (FIG. 4). In this first mode of viewing the pupil of the eye may be viewed, particularly to assist with alignment of the camera. In this mode the adaptor lens assembly can be moved into the image axis and used to change the effective focal length of the camera. By this means the portion of the eye in focus can be changed without having to move the camera 1.

When an image is to be captured the adaptor lens assembly 72 is moved out of the imaging axis optical path, the infrared protection filter 86 is moved in front of the xenon flash tube and the xenon flash tube is triggered.

In a second mode of viewing the halogen light source 80 is used and the selectable filter 82 is moved to the white or visible light pass filter and in this arrangement the eye 60 is illuminated with visible light and the digital camera sensor 66 collects the visible light to show an image of the eye on the display screen 50.

In a third mode of viewing and observing the halogen light source is used to pass a white light as in the previous embodiment but the mirror 90 is swung into the imaging axis and the image of the fundus of the eye can be viewed using the eyepiece assembly 27 with a viewer's eye 64.

The first mode of photography, however, is done with the xenon flash lamp 86 which is at conjugate point 87. In this arrangement the infra-red/visible selectable filter 82 is placed in the visible light source position 86 to ensure that there is no infra-red in the illumination and the eyepiece mirror 90 is swung out of the way so that the digital camera sensor 66 can collect the image. The flash is of such a short duration that the iris of the patient's eye 60 does not close up so a clear image of the eye can be taken. It will be noted that in the photography stage the selectable filter 82 is in the visible light passing mode so that all infrared light in the xenon flash lamp is cut out thereby preventing damage to the user's eye.

A second mode of photography is for photographing the anterior of a patient's eye. The halogen lamp 80 and the xenon lamp 85 are switched off and the adaptor lens assembly 72 is placed in position on the imaging axis 68 and illumination from the slit lamp 17 of the slit lamp is used.

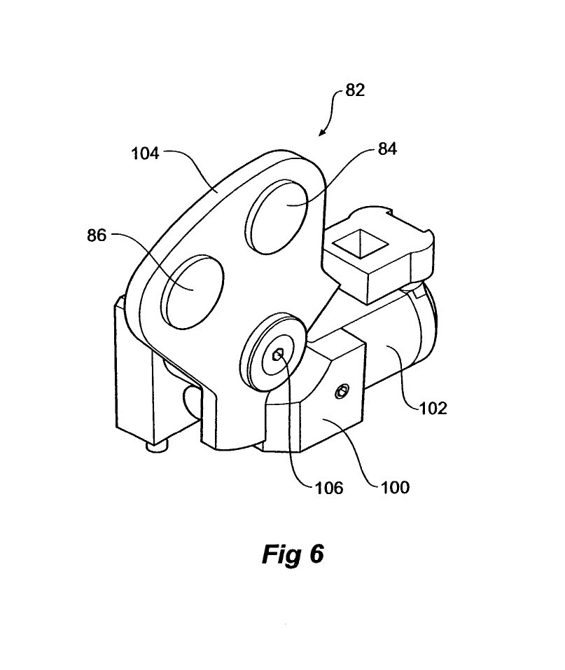

One embodiment of an adjustable or selectable filter which can be used to select either infra-red or visible light is shown in FIG. 6.

The adjustable or selectable filter device consists of a body 100 with a motor 102 adapted to rotate a plate 104 about an axis 106. Within apertures on the plate 102 is the infra-red pass filter 84 and a visible pass filter 86. By activating the motor 102 the filter plate 104 can be moved to either allow passing of only infrared light or passing of only visible light.

FIG. 7A shows the spectral response of the pixels which make up one embodiment of an image sensor suitable for the digital camera sensor 66 of the present invention.

In the image sensor for which the spectral response is shown there are four different types of pixels, being red pixels, green A pixels, green B pixels and blue pixels. The spectral response of the red pixels is shown by line 110 in the graph, the spectral response of the green A pixels is shown by the line 112, the spectral response of the green B pixels is shown by the line 114 and the spectral response of the blue pixels is shown by the line 116.

In FIG. 7B the line 120 shows the sum of the spectral responses to the image sensor as shown in FIG. 7A.

It will be noted that in the region from 450 nm to 700 nm, the visible region, the response is similar to that of the human eye making the image sensor useful for observing and recording visible images. In the region above 700 nm there is a high sensitivity to infra-red light making the same image sensor suitable for infra-red observing of images.

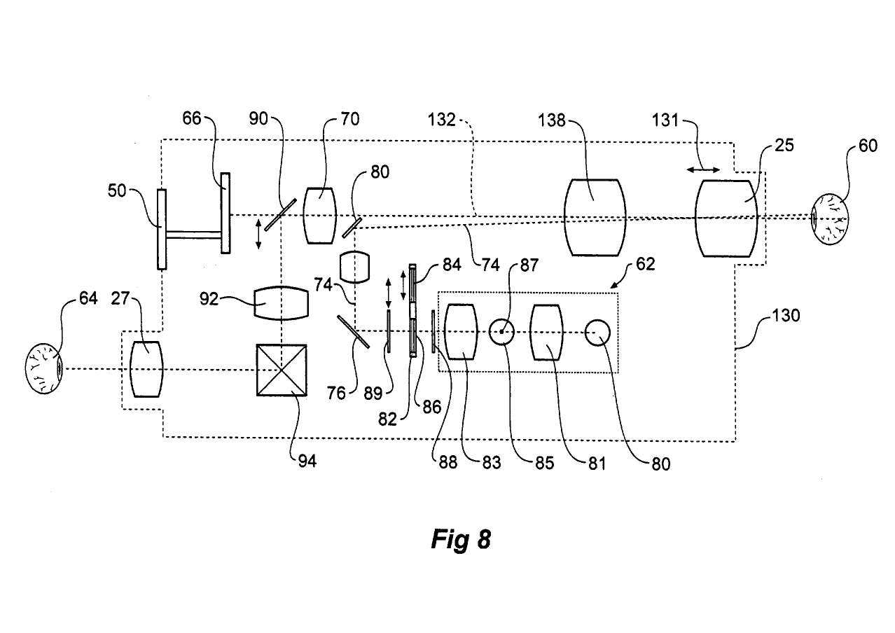

FIG. 8 shows in a schematic view the optical diagram of an eye fundus camera in a second embodiment according to the invention. Those items with the same function as in FIG. 5 have the same reference numerals.

The eye fundus camera 130 is used to view a patient's eye 60, after it has been illuminated by means of an illumination source 62, either through an eyepiece lens assembly 27 to a viewer's eye 64 or through a digital camera sensor 66 in the same manner as the earlier embodiment.

In this embodiment the imaging axis 132 includes an objective lens assembly 25, a relay lens assembly 138 and an imaging lens assembly 70 to direct light onto the sensor 66. Between the relay lens assembly 138 and the imaging lens assembly 70 a mirror 80 injects light adjacent to the imaging axis 132 on an axis 74 which is at a slight angle to the imaging axis 132 but so that the light passes through the same optical elements, the relay lens assembly 138 and the objective lens assembly 25, of the imaging axis into the eye of a patient 60.

In this embodiment the position of the objective lens assembly 25 is adjustable along the imaging axis 132, as shown by arrow 131, to allow for the existing refractive index of a patient's eye so that an accurate image of the retina of the eye can be obtained.

Illumination from the illumination assembly 62 is provided on an illumination axis 74 which is reflected first on mirror 76, through illumination lens assembly 78 and then another mirror 80. The mirror 80 is off-set from the imaging axis 132 and directs light through the objective lens 25 to illuminate the patient's eye 60. The illumination axis 74 after the mirror 80 is at a slight angle to the imaging axis 132 so that less reflections of the light source are obtained from the cornea and lens of the patient's eye which may cause a problem with unwanted light on the digital sensor 66.

The illumination arrangement 62 includes a halogen light source 80, a first condensing lens 81 and a second condensing lens 83. The halogen light source 80 is used for either visible or infrared illumination of the eye for alignment of the camera and visual observation using the digital camera as will be discussed later. For flash photography a xenon flash lamp 85 is used as discussed earlier. The xenon flash lamp 85 is placed between the first condensing lens 81 and the second condensing lens 83 at or near a conjugate point of the halogen light source 80. As both the halogen lamp 80 and the xenon lamp 85 can produce harmful ultra-violet radiation a permanent UV filter 88 is provided in the illumination axis. As the camera, according to this invention, is adapted for both infrared and visible light viewing a filter assembly 82 includes two filters on a movable base so that either infrared light using filter 84 can be passed or visible light using filter 86 can be passed but not both, as required. A cyan subtractive or red-free filter 89 may also be provided for particular photography when a user wishes to have no red in the light source.

Between the imaging lens assembly 70 and the digital camera sensor 66 is a movable mirror 90 which can be swung into the imaging axis to direct light, which would normally have passed to the digital camera sensor 66, through an eye piece lens assembly 92 and inverting prisms 94 to the eye piece lens 27. The inverting prisms put the image of the fundus of the eye into a correct orientation for viewing by a practitioner. This enables a practitioner to directly view the fundus of the eye when visible light is being used.

The operation of this embodiment of fundus camera is the same as that for the first embodiment.

Generally it will be seen that by this invention by using an image sensor which has sensitivity to a wide range of the spectrum and a single light source for both infra-red and visible light viewing, a simpler eye fundus camera can be produced.

Throughout this specification various indications have been given as to the scope of the invention but the invention is not limited to any one of these but may reside in two or more of these combined together. The examples are given for illustration only and not for limitation.

1. An eye fundus imaging apparatus having a digital camera sensor adapted to receive an image of the fundus of the eye wherein the digital camera sensor is adapted to be responsive to both the infra-red and visible spectral bands utilised in the apparatus.

2. An eye fundus imaging apparatus having a sensor arrangement, an illumination arrangement including a single light source adapted to provide both infra-red and visible light and a selector to select either the infra-red or visible light and wherein the sensor arrangement is a single digital camera sensor adapted to be sensitive to both visible and infra-red light.

3. An eye fundus imaging apparatus as in claim 2 wherein the single light source is a halogen light source which provides both the visible and infra-red light.

4. An eye fundus imaging apparatus as in claim 2 wherein the light source includes a fixed UV filter to remove ultraviolet emitted from the light to protect a patient's eye from incident wavelengths below 400 nm.

5. An eye fundus imaging apparatus having an imaging axis between an objective lens assembly and a sensor arrangement, an illumination injection device between the objective lens assembly and the sensor arrangement and an illumination apparatus to provide illumination to the illumination injection device wherein the illumination apparatus includes a single light source adapted to provide both infra-red and visible light and a selector to select either the infra-red or visible light.

6. An eye fundus imaging apparatus as in claim 5 wherein the single light source is a halogen light source which provides both the visible and infra-red light and the selector includes selectable filters to transmit either visible or infra-red light.

7. An eye fundus imaging apparatus as in claim 5 further including a flash lamp close to or at an optical conjugate to the halogen light source.

8. An eye fundus imaging apparatus as in claim 7 wherein the flash lamp is a xenon lamp.

9. An eye fundus imaging apparatus as in claim 5 wherein the illumination injection device is a prism or a mirror.

10. An eye fundus imaging apparatus as in claim 5 wherein the illumination injection device is offset from the imaging axis to provide an illumination axis which is at a slight angle to the imaging axis and to meet the imaging axis approximately at the pupil of a patients eye for efficient illumination of the patients eye.

11. An eye fundus imaging apparatus as in claim 5 wherein the illumination injection device is positioned outside the image area between the objective lens assembly and the sensor arrangement.

12. An eye fundus imaging apparatus as in claim 5 further including a relay lens assembly between the objective lens assembly and the sensor arrangement.

13. An eye fundus imaging apparatus as in claim 5 further including an imaging lens assembly between the objective lens assembly and the sensor arrangement.

14. An eye fundus imaging apparatus as in claim 5 further including a movable mirror in the imaging axis, the movable mirror when extending into the imaging axis being arranged to direct an image from the patient's eye to an eyepiece on an eyepiece axis.

15. An eye fundus imaging apparatus as in claim 14 wherein the eyepiece axis may include inverting prisms so that an upright image of the eye can be viewed in the eyepiece.

16. An eye fundus imaging apparatus as in claim 5 further including a fixed filter associated with the illumination apparatus to remove ultraviolet emitted from the light source to protect a patient's eye from incident wavelengths below 400 nm.

17. An eye fundus imaging apparatus as in claim 5 further including red free filter or cyan subtractive filter associated with the illumination apparatus.

18. An eye fundus imaging apparatus as in claim 5 wherein the sensor arrangement is a single digital camera sensor to be sensitive to both visible and infra-red light.

19. An eye fundus imaging apparatus as in claim 5 further including adjustment means to electronically adjust the position of at least one lens of the objective lens assembly, the relay lens assembly or the imaging lens assembly whereby to adjust the imaging apparatus for variations in the refractive error of the subject eye under investigation.

20. An eye fundus imaging apparatus as in claim 19 wherein the adjustment means adjusts the imaging lens assembly to adapt for a patient's refractive error.

21. An eye fundus imaging apparatus as in claim 5 further including an adaptor lens assembly on the imaging axis between the objective lens and the imaging lens wherein the adaptor lens may be moveable between a position on the imaging axis to a position outside the imaging axis wherein the adaptor lens assembly changes the focus point of the objective lens from the fundus of the eye to the anterior of the eye.

22. An eye fundus imaging apparatus as in claim 5 adapted to be mounted to an ophthalmic slit lamp assembly thereby making it usable with existing equipment in a practitioners consulting rooms.

23. An eye fundus imaging apparatus as in claim 5 further including a remote manual or foot operated switch arrangement for the fundus camera of the present invention thereby providing a practitioner with ease of operation of the camera.

24. An eye fundus imaging apparatus having an imaging axis between an objective lens assembly and a sensor arrangement, an imaging lens assembly between the objective lens assembly and the sensor arrangement and an illumination injection device positioned between the objective lens assembly and the imaging lens assembly and offset from the imaging axis and set to provide an illumination axis which is at a slight angle to the imaging axis and to focus approximately at the pupil of a patients eye, a lamp to provide illumination to the illumination injection device and a selector to select either the infra-red or visible light, wherein the lamp includes a single light source adapted to provide both infra-red and visible light, wherein the selector includes selectable filters to transmit either visible or infra-red light and wherein the sensor arrangement is a single digital camera sensor adapted to be sensitive to both visible and infra-red light.

25. An eye fundus imaging apparatus as in claim 24 wherein the single light source is an halogen light source which provides both the visible and infra-red light.

26. An eye fundus imaging apparatus as in claim 24 further including a flash lamp at a focus point of the halogen light source.

27. An eye fundus imaging apparatus having an imaging axis between an objective lens assembly and a sensor arrangement, an imaging lens assembly between the objective lens assembly and the sensor arrangement and an illumination injection device positioned between the objective lens assembly and the imaging lens assembly and offset from the imaging axis and set to provide an illumination axis which is at a slight angle to the imaging axis and to focus approximately at the pupil of a patients eye, a movable mirror between the imaging lens assembly and the sensor arrangement, the movable mirror when extending into the imaging axis being arranged to direct an image from the patient's eye to an eyepiece on an eyepiece axis, a lamp to provide illumination to the illumination injection device and a selector to select either infra-red or visible light from the lamp, a flash lamp close to or at an optical conjugate to the lamp to provide illumination for photography, wherein the lamp includes a single light source adapted to provide both infra-red and visible light, wherein the selector includes selectable filters to transmit either visible or infra-red light and wherein the sensor arrangement is a single digital camera sensor adapted to be sensitive to both visible and infra-red light.

28. An ophthalmic slit lamp assembly having a fundus camera mounted thereon.

29. An ophthalmic slit lamp assembly having a fundus camera mounted to a tonometer adaptor on the ophthalmic slit lamp assembly, the fundus camera including an alignment mode in which a joystick on the ophthalmic slit lamp assembly is used to vary the position of the fundus camera until the anterior of the eye is in focus at which stage the camera is at the correct distance from the eye for fundus photography.

30. An ophthalmic slit lamp assembly as in claim 29 wherein the fundus camera includes at least one red LED whereby in the alignment mode illumination of the anterior of the eye is by using the at least one red LED.
